(19) United States
(12) Patent Application Publication
(10) Pub. No.: US 2023/0248695 A1
(43) Pub. Date: Aug. 10, 2023

(54) COMBINATIONS OF CANCER THERAPEUTICS

(71) Applicant: Merck Patent GmbH, Darmstadt (DE)

(72) Inventors: Manja FRIESE-HAMIM, Darmstadt (DE); Felix Rohdich, Darmstadt (DE); Claude Gimmi, Darmstadt (DE); Christoph Schultes, Maroussi (GR); Olga Bogatyrova, Darmstadt (DE)

(73) Assignee: Merck Patent GmbH, Darmstadt (DE)

(21) Appl. No.: 18/004,314

(22) PCT Filed: Jul. 6, 2021
(86) PCT No.: PCT/EP2021/068564

§ 371 Date: Jan. 5, 2023

(30) Foreign Application Priority Data
Jul. 9, 2020 (EP) 20184962.7

(51) Int. Cl. A61K 31/404; A61K 31/47; A61K 31/4439; A61P 35/00

(52) U.S. Cl. CPC A61K 31/404; A61K 31/47; A61K 31/4439; A61P 35/00

(57) ABSTRACT

Combinations of (S)-3-Hydroxy-1-(1H-indol-5-yl)-2-oxo-pyrrolidine-3-carboxylic acid 3,5-difluoro-benzylamide and a VEGFR/VEGF inhibitor, or their physiologically acceptable salts are disclosed. Such combination are useful for the prophylaxis or treatment of cancer.

### TECHNICAL FIELD

The invention relates to combinations of (S)-3-Hydroxy-1-(1H-indol-5-yl)-2-oxo-pyrrolidine-3-carboxylic acid 3,5-difluoro-benzylamide (hereinafter referred to as Compound A) and an inhibitor of one or more of VEGFR1, VEGFR2, VEGFR3 and VEGF (hereinafter referred to as a VEGFR/VEGF inhibitor), or their physiologically acceptable salts, as well as to the use of such combinations for the prophylaxis or treatment of cancer.

### BACKGROUND

In 1990, Judah Folkman’s laboratory published on the anti-angiogenic activity of a natural compound named fumagillin, which had been isolated in 1951 from the fungus Aspergillus fumigatus (Ingber et al., Nature 1990;348(6301):555-7). The molecular target of fumagillin and derivatives was identified in 1997 as methionine aminopeptidase 2 (MetAP2) by the laboratory of Craig Crews (Sin et al., Proc Natl Acad Sci USA 1997;94:6099-103). Methionine aminopeptidases are enzymes which remove the amino terminal amino acid methionine from proteins during or after the translation process. Methionine is cleaved from some but not all cellular proteins, especially proteins bearing a small amino acid at the second position. As part of protein maturation, cleavage is assumed to be relevant for protein stability (N-end rule) and proper function (protein folding, N-terminal myristoylation).

Three MetAPs can be found in humans, MetAP1, MetAP2, and MAP1D. MetAP2 is widely expressed in all human tissues, although differences exist in expression levels based on mRNA expression data. Complete knockout of MetAP2 in mice is embryonic lethal with a pronounced block at the gastrulation stage. A tissue-restricted knockout in the vascular endothelial cell compartment was embryonic lethal and showed a pronounced abnormality in vascular development (Yeh et al., Proc Natl Acad Sci USA 2006; 103(27): 10379-84.), indicating that MetAP2 is important during development and required for the formation of the vascular system.

Pharmacological inhibition of MetAP2 using different classes of inhibitors has been shown to block proliferation of vascular endothelial cells, explaining, at least in part, the anti-angiogenic mechanism of action (Wang et al., Proc Natl Acad Sci USA 2008;105(6):1838-43.; Bernier et al., J Cell Biochem 2005;95(6):1191-203). Subsequently, anti-proliferative activity on tumor cell lines has been demonstrated indicating potential for direct anti-tumor effects (Wang et al., Cancer Res 2003;63(22):7861-9; Wang et al., Proc Natl Acad Sci USA 2008;105(6):1838-43).

The discovery of fumagillin with potent anti-angiogenic and anti-proliferative activities promoted the development of MetAP2 antagonists as a novel class of anticancer agents. MetAP2 plays an important role in the development of different types of cancer. MetAP2 inhibition leads to a delay in cell cycle progression in endothelial cells and in a subset of tumor cells. Thereby, MetAP2 inhibitors block neo-angiogenesis both in vitro and in vivo and show potent antitumor efficacy in a variety of tumor types of human origin in mouse models.

In clinical trials fumagillin proved unsuitable as an anticancer agent due to its pronounced neurotoxicity. Compound A is a potent, reversible, non-covalent and orally bioavailable inhibitor of MetAP2 (see also Heinrich et al., J Med Chem. 2019;62(24):11119-11134. doi:10.1021/acs.jmedchem.9b01070). Compound A is not chemically related to fumagillin and has not shown signs of neurotoxicity in toxicology studies. Compound A has been found to inhibit growth of endothelial cells, murine and human tumor cells as well as patient-derived tumors. Compound A has demonstrated antiangiogenic and antitumoral activity in mouse models. It has been shown that MetAP2 can be effectively inhibited using Compound A, as evidenced by the accumulation of an unprocessed MetAP2 substrate, methionylated elongation factor 1 α, Met-EF1α. In comparison to irreversible MetAP2 inhibitors, Compound A has the promise of an improved safety and tolerability profile, which is beneficial for administration over extended periods. Compound A, processes for its preparation, as well as its use in the treatment of cancer, are disclosed in WO 2013/149704 A1 (Compound A is referred to as “B8” in WO 2013/149704 A1); see also WO 2012/048775 A1. Also, an asymmetric synthesis of Compound A is disclosed in Heinrich et al. J Med Chem. 2019;62(24):11119-11134. doi:10.1021/acs.jmedchem.9b01070.

An objective of the present invention was to find ways to further advance the pharmaceutical utility of Compound A. In this context, combinations of Compound A with VEGFR/VEGF inhibitors were studied.

Known VEGFR/VEGF inhibitors include aflibercept, apatinib, axitinib, bevacizumab, brivanib alaninate, cabozantinib, cediranib, lenvatinib, linifanib, pazopanib, ponatinib, ramucirumab, regorafenib, sorafenib, sunitinib, vandetanib, and the anti-VEGFR2 antibody 33C3.

Cabozantinib (S)-malate is a tyrosine kinase inhibitor that initially received Food and Drug Administration (FDA) approval as a second-line therapy for patients with metastatic renal cell carcinoma (mRCC) who developed resistance to first-line agents; cabozantinib (S)-malate has more recently received approval as a first-line therapy. Cabozantinib targets, among other kinases, VEGF receptors (VEGFR) 1 to 3. The recommended dosage of cabozantinib (S)-malate in mRCC is 60 mg once daily without food until the patient no longer experiences clinical benefit or experiences unacceptable toxicity (see e.g. the label for the commercial cabozantinib (S)-malate product Cabometyx of January 2020, as published on the website of FDA: https://www.accessdata.fda.gov/drugsatfda_docs/label/2020/208692s007lbl.pdf). Cabozantinib is commercially available, for instance in the form of the (S)-malate salt of cabozantinib (i.e. cabozantinib (S)-malate).

Axitinib is another tyrosine kinase inhibitor. Information on axitinib may be found e.g. in the label for the commercial axitinib product INLYTA of June 2020, as published on the website of FDA (https://www.accessdata.fda.gov/drugsatfda_docs/label/2020/202324s011lbl.pdf): Axitinib has been shown to inhibit receptor tyrosine kinases including VEGFR-1, VEGFR-2, and VEGFR-3 at therapeutic plasma concentrations. VEGF-mediated endothelial cell proliferation and survival were inhibited by axitinib in vitro and in mouse models. Axitinib was shown to inhibit tumor growth and phosphorylation of VEGFR-2 in tumor xenograft mouse models. Axitinib has been approved e.g. as a single agent for the treatment of advanced renal cell carcinoma (RCC) after failure of one prior systemic therapy. In combination with avelumab, axitinib is indicated for the first-line treatment of patients with advanced renal cell carcinoma (RCC). In combination with pembrolizumab, axitinib is indicated for the first-line treatment of patients with advanced renal cell carcinoma. Axitinib is commercially available, for instance in the form of a free base.

In first-line treatment of advanced RCC, the recommended dose of axitinib is 5 mg orally taken twice daily (12 hours apart) with or without food in combination with avelumab 800 mg administered as an intravenous infusion over 60 minutes every 2 weeks until disease progression or unacceptable toxicity. When axitinib is used in combination with avelumab, dose escalation of axitinib above the initial 5 mg dose may be considered at intervals of two weeks or longer. The recommended dose of axitinib is 5 mg orally twice daily (12 hours apart) with or without food in combination with pembrolizumab 200 mg every 3 weeks or 400 mg every 6 weeks administered as an intravenous infusion over 30 minutes until disease progression or unacceptable toxicity. When axitinib is used in combination with pembrolizumab, dose escalation of axitinib above the initial 5 mg dose may be considered at intervals of six weeks or longer.

In second-line treatment of advanced RCC, when axitinib is used as a single agent, the recommended starting oral dose is 5 mg twice daily. In this context, axitinib doses are to be administered approximately 12 hours apart with or without food.

The proangiogenic signaling molecule VEGF and its receptors VEGFR1, VEGFR2, and VEGFR3 play key roles in tumor development. These receptors are implicated in pathologic angiogenesis, tumor growth, and cancer progression. Induction of angiogenesis has been increasingly recognized as a crucial step in tumor progression and is one of the hallmarks of cancerous growth. For instance, angiogenesis is an important factor for cancer development and progression in renal cell carcinoma (RCC). RCC represents a heterogeneous group of cancers that arise from the kidney. The most common histological variant of RCC is clear cell RCC, which comprises about 70% of RCC and has the highest metastatic potential. Clear cell RCC is characterized by inactivation of the Von-Hippel Lindau (VHL) tumor suppressor gene, which results in hyperactivity of hypoxia-inducible factor-α (HIFα). This leads to a production of angiogenic factors, such as VEGF and platelet-derived growth factor. The activity of these factors is associated with oncogenesis, growth, and metastatic potential of RCC.

Medical treatment for metastatic RCC (mRCC) has expanded considerably from a target of rapamycin (mTOR). Today the salvage approach has evolved, to include immune checkpoint blockade (ICB) targeting programmed death-1 (PD-1) and programmed death-ligand 1 (PD-L1) (Salgia et al., Curr Treat Options Oncol 2019;20(5):41.). VEGFR TKIs alone, ICB, or VEGFR TKIs combined with ICB are options for first-line treatment of mRCC. Cabozantinib, nivolumab, and axitinib are subsequent therapy options. The tyrosine kinase inhibitors cabozantinib and axitinib are thus among the standard-of-care (SoC) agents for the treatment of RCC.

Still, there remains a need for new treatment options for cancer, including RCC. The present invention addresses this and other needs in the art.

### SUMMARY OF THE INVENTION

Surprisingly, it has been found by the inventors of the present patent application that Compound A achieves a significant combination benefit versus monotherapy when combined with cabozantinib or axitinib in the treatment of RCC patient derived xenografts (PDX). The inventors’ findings suggest that Compound A in combination with VEGFR/VEGF inhibitors such as axitinib or cabozantinib can be used to successfully treat cancer, including RCC.

The present invention relates to combinations of Compound A and a VEGFR/VEGF inhibitor such as cabozantinib or axitinib, or their physiologically acceptable salts, as well as to the use of such combinations for the prophylaxis or treatment of cancer, including RCC. The present invention relates to the embodiments described in the claims.

The combinations and their uses according to the invention, as summarized above and described further below, result in surprising significant combination benefits in cancer therapy, including therapy of RCC. This is shown in the Examples further below.

### BRIEF DESCRIPTION OF THE DRAWINGS

FIG. 1: Treatment with vehicle or Compound A and/or cabozantinib: Progression-free survival of mice bearing RCC patient derived xenografts (PDX), represented as Kaplan-Meier curve. Number of days required to reach 173% relative tumor volume (RTV) (censoring at latest evaluation if endpoint not reached). CompA, compound A.

FIG. 2: Treatment with vehicle or Compound A and/or cabozantinib: Overall survival of mice bearing RCC patient derived xenografts (PDX), represented as Kaplan-Meier curve. Number of days required to reach 1000% RTV (censoring at latest evaluation if endpoint not reached). CompA, compound A.

FIG. 3: Treatment with vehicle or Compound A and/or cabozantinib: Body weight change of mice bearing RCC patient derived xenografts (PDX); one exemplary PDX model shown per treatment group.

FIG. 4: Treatment with vehicle or Compound A and/or axitinib: Progression-free survival of mice bearing RCC patient derived xenografts (PDX), represented as Kaplan-Meier curve. Number of days required to reach 173% RTV (censoring at latest evaluation if endpoint not reached). CompA, compound A.

FIG. 5: Treatment with vehicle or Compound A and/or axitinib: Overall survival of mice bearing RCC patient derived xenografts (PDX), represented as Kaplan-Meier curve. Number of days required to reach 1000% RTV (censoring at latest evaluation if endpoint not reached). CompA, compound A.

FIG. 6: Treatment with vehicle or Compound A and/or axitinib: Body weight change of mice bearing RCC patient derived xenografts (PDX); one exemplary PDX model shown per treatment group.

### DETAILED DESCRIPTION OF THE INVENTION

The present invention relates to combinations of Compound A and a VEGFR/VEGF inhibitor, or their physiologically acceptable salts, as well as to the use of such combinations for the prophylaxis or treatment of cancer, as described herein above and below.

The present invention relates to a compound mixture comprising the following compounds a) and b):
a) Compound A or a physiologically acceptable salt thereof;
b) a VEGFR/VEGF inhibitor or a physiologically acceptable salt thereof.

In some embodiments of the invention, the VEGFR/VEGF inhibitor is selected from the group consisting of aflibercept, apatinib, axitinib, bevacizumab, brivanib alaninate, cabozantinib, cediranib, lenvatinib, linifanib, pazopanib, ponatinib, ramucirumab, regorafenib, sorafenib, sunitinib, vandetanib, and 33C3. In some embodiments of the invention, the VEGFR/VEGF inhibitor is cabozantinib or axitinib. In some specific embodiments, the VEGFR/VEGF inhibitor is cabozantinib, e.g. cabozantinib (S)-malate. In other specific embodiments, the VEGFR/VEGF inhibitor is axitinib.

Cabozantinib is also referred to as N-{4-[(6,7-dimethoxyquinolin-4-yl)oxy]phenyl}-N′-(4-fluorophenyl)cyclopropane-1,1-dicarboxamide. Cabozantinib (S)-malate is also referred to as N-{4-[(6,7-dimethoxyquinolin-4-yl)oxy]phenyl}-N′-(4-fluorophenyl)cyclopropane-1,1-dicarboxamide, (2S)-hydroxybutane-dioate.

Axitinib is also referred to as N-methyl-2-({3-[(1E)-2-(pyridin-2-yl)ethenyl]-1H-indazol-6-yl}sulfanyl)benzamide.

In some embodiments, the present invention thus relates to a compound mixture comprising the following compounds a) and b):
a) Compound A or a physiologically acceptable salt thereof;
b) cabozantinib or axitinib, or a physiologically acceptable salt thereof.

In some embodiments, present invention relates to a compound mixture comprising the following compounds a) and b):
a) Compound A or a physiologically acceptable salt thereof;
b) cabozantinib or a physiologically acceptable salt thereof.

In some embodiments, present invention relates to a compound mixture comprising the following compounds a) and b):
a) Compound A or a physiologically acceptable salt thereof;
b) cabozantinib (S)-malate.

In some embodiments, present invention relates to a compound mixture comprising the following compounds a) and b):
a) Compound A or a physiologically acceptable salt thereof;
b) axitinib or a physiologically acceptable salt thereof.

Moreover, the present invention relates to a pharmaceutical composition, comprising a compound mixture of the active pharmaceutical ingredients (API’s) Compound A and a VEGFR/VEGF inhibitor or their respective physiologically acceptable salts, and optionally further comprising one or more excipient(s) and/or adjuvant(s). In some embodiments of the invention, the VEGFR/VEGF inhibitor comprised by the pharmaceutical composition is selected from the group consisting of aflibercept, apatinib, axitinib, bevacizumab, brivanib alaninate, cabozantinib, cediranib, lenvatinib, linifanib, pazopanib, ponatinib, ramucirumab, regorafenib, sorafenib, sunitinib, vandetanib, and 33C3. In some embodiments of the invention, the VEGFR/VEGF inhibitor is cabozantinib or axitinib. In some specific embodiments, the VEGFR/VEGF inhibitor is cabozantinib, e.g. cabozantinib (S)-malate. In other specific embodiments, the VEGFR/VEGF inhibitor is axitinib.

The present invention relates to a pharmaceutical composition comprising any one of the compound mixtures according to the invention and, optionally, further comprising one or more excipient(s) and/or adjuvant(s).

For some compounds, suitable acid-addition salts are inorganic or organic salts of all physiologically or pharmacologically acceptable acids, for example halides, in particular hydrochlorides or hydrobromides, lactates, sulfates, citrates, tartrates, maleates, fumarates, oxalates, acetates, phosphates, methylsulfonates, benzoates or p-toluenesulfonates.

A preferred form of Compound A is its free base. Salt forms of Compound A include its sodium, potassium, calcium or magnesium salts.

The pharmaceutical compositions according to the invention comprise mixtures of the two API’s, for example in the ratio 1:1, 1:2, 1:3, 1:4, 1:5, 1:10, 1:100 or 1:1000.

The pharmaceutical composition may furthermore comprise at least one solid, liquid and/or semi-liquid excipient and/or adjuvant. Therefore, the invention also relates to a pharmaceutical composition comprising the said API mixture according to the invention and the said excipients and/or adjuvants.

The present invention also relates to a set (also referred to as a kit) comprising separate packs of the following compounds a) and b):
a) Compound A or a physiologically acceptable salt thereof;
b) a VEGFR/VEGF inhibitor or a physiologically acceptable salt thereof.

For instance, the present invention relates to a set (also referred to as a kit) comprising separate packs of the following components:
a) a pharmaceutical composition comprising Compound A or a physiologically acceptable salt thereof,
b) a pharmaceutical composition comprising a VEGFR/VEGF inhibitor or a physiologically acceptable salt thereof and, optionally,
c) a pharmaceutical composition comprising a third cancer therapeutic.

In some embodiments of the invention, the VEGFR/VEGF inhibitor in the set (kit) is selected from the group consisting of aflibercept, apatinib, axitinib, bevacizumab, brivanib alaninate, cabozantinib, cediranib, lenvatinib, linifanib, pazopanib, ponatinib, ramucirumab, regorafenib, sorafenib, sunitinib, vandetanib, and 33C3. In some embodiments of the invention, the VEGFR/VEGF inhibitor is cabozantinib or axitinib. In some specific embodiments, the VEGFR/VEGF inhibitor is cabozantinib, e.g. cabozantinib (S)-malate. In other specific embodiments, the VEGFR/VEGF inhibitor is axitinib.

The set comprises suitable containers, such as boxes, individual bottles, bags or ampoules. The set may, for example, comprise separate ampoules, each containing a pharmaceutical composition comprising an effective amount of Compound A and/or pharmaceutically acceptable salts thereof, a pharmaceutical composition comprising an effective amount of the VEGFR/VEGF inhibitor (e.g. cabozantinib or axitinib) and/or pharmaceutically acceptable salts thereof and, optionally, a pharmaceutical composition comprising an effective amount of a third cancer therapeutic in dissolved or lyophilised form.

Cancer therapeutics that can be combined as a “third cancer therapeutic” with Compound A and the VEGFR/VEGF inhibitor (e.g. cabozantinib or axitinib), according to the invention, may include one or more, but preferably one, of the following agents:
Alkylating agents, such as altretamine, bendamustine, busulfan, carmustine, chlorambucil, chlormethine, cyclophosphamide, dacarbazine, ifosfamide, improsulfan tosilate, lomustine, melphalan, mitobronitol, mitolactol, nimustine, ranimustine, temozolomide, thiotepa, treosulfan, mechloretamine, carboquone, apaziquone, fotemustine, glufosfamide, palifosfamide, pipobroman, trofosfamide, uramustine;
Platinum Compounds, such as carboplatin, cisplatin, eptaplatin, miriplatine hydrate, oxaliplatin, lobaplatin, nedaplatin, picoplatin, satraplatin;
DNA altering agents, such as amrubicin, bisantrene, decitabine, mitoxantrone, procarbazine, trabectedin, clofarabine, amsacrin, brostallicin, pixantrone, laromustine;
Topoisomerase inhibitors, such as etoposide, irinotecan, razoxane, sobuzoxane, teniposide, topotecan, amonafide, belotecan, elliptinium acetate, voreloxin;
Microtubule modifiers, such as cabazitaxel, docetaxel, eribulin, ixabepilone, paclitaxel, vinblastine, vincristine, vinorelbine, vindesine, vinflunine, fosbretabulin, tesetaxel:
Antimetabolites, such as asparaginase, azacitidine, calcium levofolinate, capecitabine, cladribine, cytarabine, enocitabine, floxuridine, fludarabine, fluorouracil, gemcitabine, mercaptopurine, methotrexate, nelarabine, pemetrexed, pralatrexate, azathioprine, thioguanine, carmofur, doxifluridine, elacytarabine, raltitrexed, sapacitabine, tegafur, trimetrexate;
Anticancer antibiotics, such as bleomycin, dactinomycin, doxorubicin, epirubicin, idarubicin, levamisole, miltefosine, mitomycin C, romidepsin, streptozocin, valrubicin, zinostatin, zorubicin, daunurobicin, plicamycin, aclarubicin, peplomycin, pirarubicin;
Hormones/Antagonists, such as abarelix, abiraterone, bicalutamide, buserelin, calusterone, chlorotrianisene, degarelix, dexamethasone, estradiol, fluocortolone, fluoxymesterone, flutamide, fulvestrant, goserelin, histrelin, leuprorelin, megestrol, mitotane, nafarelin, nandrolone, nilutamide, octreotide, prednisolone, raloxifene, tamoxifen, thyrotropin alfa, toremifene, trilostane, triptorelin, diethylstilbestrol, acolbifene, danazol, deslorelin, epitiostanol, orteronel, enzalutamide;
Aromatase inhibitors, such as aminoglutethimide, anastrozole, exemestane, fadrozole, letrozole, testolactone, formestane;
Small molecule kinase inhibitors, such as crizotinib, dasatinib, erlotinib, imatinib, lapatinib, nilotinib, pazopanib, regorafenib, ruxolitinib, sorafenib, sunitinib, vandetanib, vemurafenib, bosutinib, gefitinib, afatinib, alisertib, dabrafenib, dacomitinib, dinaciclib, dovitinib, enzastaurin, nintedanib, lenvatinib, linifanib, linsitinib, masitinib, midostaurin, motesanib, neratinib, orantinib, perifosine, ponatinib, radotinib, rigosertib, tipifarnib, tivantinib, tivozanib, trametinib, pimasertib, brivanib alaninate, cediranib, apatinib, carfilzomib, ibrutinib, icotinib;
Photosensitizers such as methoxsalen, porfimer sodium, talaporfin, temoporfin;
Cytokines, such as aldesleukin, interferon alfa, interferon alfa2a, interferon alfa2b, tasonermin, teceleukin, oprelvekin;
Drug conjugates, such as denileukin diftitox, ibritumomab tiuxetan, iobenguane I123, prednimustine, trastuzumab emtansine, estramustine, gemtuzumab ozogamicin, aflibercept, cintredekin besudotox, edotreotide, inotuzumab ozogamicin, naptumomab estafenatox, oportuzumab monatox, technetium (99mTc) arcitumomab, vintafolide;
Vaccines, such as sipuleucel, vitespen, emepepimut-S, oncoVAX, rindopepimut, troVax, stimuvax;
Miscellaneous agents, such as alitretinoin, bexarotene, bortezomib, everolimus, ibandronic acid, imiquimod, lenalidomide, lentinan, metirosine, mifamurtide, pamidronic acid, pegaspargase, pentostatin, sipuleucel, sizofiran, tamibarotene, temsirolimus, thalidomide, tretinoin, vismodegib, zoledronic acid, thalidomide, vorinostat, celecoxib, cilengitide, entinostat, etanidazole, ganetespib, idronoxil, iniparib, ixazomib, lonidamine, nimorazole, panobinostat, peretinoin, plitidepsin, pomalidomide, procodazol, ridaforolimus, tasquinimod, telotristat, thymalfasin, tirapazamine, tosedostat, trabedersen, ubenimex, valspodar, gendicine, picibanil, reolysin, retaspimycin hydrochloride, trebananib, virulizin.

The compounds and compound mixtures according to the invention can be adapted for administration via any desired suitable method, for example by oral (including buccal or sublingual), rectal, nasal, topical (including buccal, sublingual or transdermal), vaginal or parenteral (including subcutaneous, intramuscular, intravenous or intradermal) methods. Such pharmaceutical compositions (also referred to as pharmaceutical preparations) can be prepared using all processes known in the pharmaceutical art by, for example, combining the active ingredient(s) with excipient(s) and/or adjuvant(s).

Compounds and compound mixtures adapted for oral administration can be administered as separate units, such as, for example, capsules or tablets; powders or granules; solutions or suspensions in aqueous or non-aqueous liquids; edible foams or foam foods; or oil-in-water liquid emulsions or water-in-oil liquid emulsions.

Thus, for example, in the case of oral administration in the form of a tablet or capsule, the compound or compound mixtures can be combined with an oral, non-toxic and pharmaceutically acceptable inert excipient, such as, for example, ethanol, glycerol, water and the like. Powders are prepared by comminuting the compound to a suitable fine size and mixing it with a pharmaceutical excipient comminuted in a similar manner, such as, for example, an edible carbohydrate, such as, for example, starch or mannitol. A flavour, preservative, dispersant and dye may likewise be present.

Capsules can be produced by preparing a powder mixture as described above and filling shaped gelatine shells therewith. Glidants and lubricants, such as, for example, highly disperse silicic acid, talc, magnesium stearate, calcium stearate or polyethylene glycol in solid form, can be added to the powder mixture before the filling operation. A disintegrant or solubiliser, such as, for example, agar-agar, calcium carbonate or sodium carbonate, may likewise be added in order to improve the availability of the compound or compound mixtures after the capsule has been taken.

In addition, if desired or necessary, suitable binders, lubricants and disintegrants as well as dyes can likewise be incorporated into the mixture. Suitable binders include starch, gelatine, natural sugars, such as, for example, glucose or beta-lactose, sweeteners made from maize, natural and synthetic rubber, such as, for example, acacia, tragacanth or sodium alginate, carboxymethylcellulose, polyethylene glycol, waxes, and the like. The lubricants used in these dosage forms include sodium oleate, sodium stearate, magnesium stearate, sodium benzoate, sodium acetate, sodium chloride and the like. The disintegrants include, without being restricted thereto, starch, methylcellulose, agar, bentonite, xanthan gum and the like. Tablets are formulated by, for example, preparing a powder mixture, granulating or dry-pressing the mixture, adding a lubricant and a disintegrant and pressing the entire mixture to give tablets. A powder mixture is prepared by mixing the compound comminuted in a suitable manner with a diluent or a base, as described above, and optionally with a binder, such as, for example, carboxymethylcellulose, an alginate, gelatine or polyvinylpyrrolidone, a dissolution retardant, such as, for example, paraffin, an absorption accelerator, such as, for example, a quaternary salt, and/or an absorbant, such as, for example, bentonite, kaolin or dicalcium phosphate. The powder mixture can be granulated by wetting it with a binder, such as, for example, syrup, starch paste, acadia mucilage or solutions of cellulose or polymer materials and pressing it through a sieve. As an alternative to granulation, the powder mixture can be run through a tableting machine, giving lumps of non-uniform shape which are broken up to form granules. The granules can be lubricated by addition of stearic acid, a stearate salt, talc or mineral oil in order to prevent sticking to the tablet casting moulds. The lubricated mixture is then pressed to give tablets. The compounds and compound mixtures according to the invention can also be combined with a free-flowing inert excipient and then pressed directly to give tablets without carrying out the granulation or dry-pressing steps. A transparent or opaque protective layer consisting of a shellac sealing layer, a layer of sugar or polymer material and a gloss layer of wax may be present. Dyes can be added to these coatings in order to be able to differentiate between different dosage units.

Oral liquids, such as, for example, solution, syrups and elixirs, can be prepared in the form of dosage units so that a given quantity comprises a prespecified amount of the compound. Syrups can be prepared by dissolving the compounds and compound mixtures in an aqueous solution with a suitable flavour, while elixirs are prepared using a non-toxic alcoholic vehicle. Suspensions can be formulated by dispersion of the compound in a non-toxic vehicle. Solubilisers and emulsifiers, such as, for example, ethoxylated isostearyl alcohols and polyoxyethylene sorbitol ethers, preservatives, flavour additives, such as, for example, peppermint oil, or natural sweeteners or saccharin or other artificial sweeteners, and the like, can likewise be added.

The dosage unit formulations for oral administration can, if desired, be encapsulated in microcapsules. The formulation can also be prepared in such a way that the release is extended or retarded, such as, for example, by coating or embedding of particulate material in polymers, wax and the like.

The compounds and compound mixtures according to the invention and salts thereof can also be administered in the form of liposome delivery systems, such as, for example, small unilamellar vesicles, large unilamellar vesicles and multilamellar vesicles. Liposomes can be formed from various phospholipids, such as, for example, cholesterol, stearylamine orphosphatidylcholines.

The compounds and compound mixtures according to the invention can also be delivered using monoclonal antibodies as individual carriers to which the compound molecules are coupled. The compounds and compound mixtures can also be coupled to soluble polymers as targeted medicament carriers. Such polymers may encompass polyvinylpyrrolidone, pyran copolymer, polyhydroxypropylmethacrylamidophenol, polyhydroxyethylaspartamidophenol or polyethylene oxide polylysine, substituted by palmitoyl radicals. The compounds may furthermore be coupled to a class of biodegradable polymers which are suitable for achieving controlled release of a medicament, for example polylactic acid, poly-epsilon-caprolactone, polyhydroxybutyric acid, polyorthoesters, polyacetals, polydihydroxypyrans, polycyanoacrylates and crosslinked or amphipathic block copolymers of hydrogels.

Compounds and compound mixtures adapted for transdermal administration can be administered as independent plasters for extended, close contact with the epidermis of the recipient. Thus, for example, the active ingredient can be delivered from the plaster by iontophoresis, as described in general terms in Pharmaceutical Research, 3(6):318, 1986.

Compounds and compound mixtures adapted for topical administration can be formulated as ointments, creams, suspensions, lotions, powders, solutions, pastes, gels, sprays, aerosols or oils.

For the treatment of the eye or other external tissue, for example mouth and skin, the formulations are preferably applied as topical ointment or cream. In the case of formulation to give an ointment, the compounds or compound mixtures can be employed either with a paraffinic or a water-miscible cream base. Alternatively, the compounds or compound mixtures can be formulated to give a cream with an oil-in-water cream base or a water-in-oil base.

Compounds and compound mixtures adapted for topical application to the eye include eye drops, in which the active ingredient is dissolved or suspended in a suitable carrier, in particular an aqueous solvent.

Compounds and compound mixtures adapted for topical application in the mouth encompass lozenges, pastilles and mouthwashes.

Compounds and compound mixtures adapted for rectal administration can be administered in the form of suppositories or enemas.

Compounds and compound mixtures adapted for nasal administration in which the carrier substance is a solid comprise a coarse powder having a particle size, for example, in the range 20-500 microns, which is administered in the manner in which snuff is taken, i.e. by rapid inhalation via the nasal passages from a container containing the powder held close to the nose. Suitable formulations for administration as nasal spray or nose drops with a liquid as carrier substance encompass active-ingredient solutions in water or oil.

Compounds and compound mixtures adapted for administration by inhalation encompass finely particulate dusts or mists, which can be generated by various types of pressurised dispensers with aerosols, nebulisers or insufflators.

Compounds and compound mixtures adapted for vaginal administration can be administered as pessaries, tampons, creams, gels, pastes, foams or spray formulations.

Compounds and compound mixtures adapted for parenteral administration include aqueous and non-aqueous sterile injection solutions comprising antioxidants, buffers, bacteriostatics and solutes, by means of which the formulation is rendered isotonic with the blood of the recipient to be treated; and aqueous and non-aqueous sterile suspensions, which may comprise suspension media and thickeners. The formulations can be administered in single-dose or multidose containers, for example sealed ampoules and vials, and stored in freeze-dried (lyophilised) state, so that only the addition of the sterile carrier liquid, for example water for injection purposes, immediately before use is necessary. Injection solutions and suspensions prepared in accordance with the recipe can be prepared from sterile powders, granules and tablets.

It goes without saying that, in addition to the above particularly mentioned constituents, the compound mixtures and pharmaceutical compositions according to the invention may also comprise other agents usual in the art with respect to the particular type of pharmaceutical formulation; thus, for example, compounds or compound mixtures which are suitable for oral administration may comprise flavours.

The pharmaceutical preparations according to the invention can be employed as medicaments in human and veterinary medicine. Suitable excipients are organic or inorganic substances which are suitable for enteral (for example oral), parenteral or topical administration and do not react with the compounds of the invention, for example water, vegetable oils, benzyl alcohols, polyethylene glycols, gelatine, carbohydrates, such as lactose or starch, magnesium stearate, talc or Vaseline. Suitable for enteral administration are, in particular, tablets, coated tablets, capsules, syrups, juices, drops or suppositories, suitable for parenteral administration are solutions, preferably oil-based or aqueous solutions, furthermore suspensions, emulsions or implants, and suitable for topical application are ointments, creams or powders. The compounds and compound mixtures may also be lyophilised and the resultant lyophilisates used, for example, for the preparation of injection preparations.

The preparations indicated may be sterilised and/or comprise adjuvants, such as lubricants, preservatives, stabilisers and/or wetting agents, emulsifiers, salts for modifying the osmotic pressure, buffer substances, dyes, flavours and/or aroma substances. They can, if desired, also comprise one or more further active ingredients, for example one or more vitamins.

In some embodiments of the present invention, Compound A is formulated as 1 mg, 5 mg or 30 mg hard gelatin capsules for oral administration; in some of these embodiments, no other excipients are used. For example, in some embodiments of the invention, the set (kit) comprises such hard gelatin capsules as the pharmaceutical composition comprising Compound A. In other embodiments of the invention, Compound A is formulated as tablets for oral administration.

In some embodiments of the present invention, the commercial cabozantinib drug product (namely cabozantinib (S)-malate) tablet formulation (Cabometyx; Exelixis, Inc.) is used; this tablet formulation is marketed in three strengths (20, 40, and 60 mg).

In some embodiments of the present invention, the commercial axitinib drug product tablet formulation (Inlyta; Pfizer) is used; this tablet formulation is marketed in two strengths (1 and 5 mg).

The present invention also relates to a method for prophylaxis and/or treatment of cancer, the method comprising administering to a subject the following compounds a) and b): a) Compound A or a physiologically acceptable salt thereof; b) a VEGFR/VEGF inhibitor or a physiologically acceptable salt thereof. In some embodiments of the invention, the VEGFR/VEGF inhibitor administered in the method is selected from the group consisting of aflibercept, apatinib, axitinib, bevacizumab, brivanib alaninate, cabozantinib, cediranib, lenvatinib, linifanib, pazopanib, ponatinib, ramucirumab, regorafenib, sorafenib, sunitinib, vandetanib, and 33C3. In some embodiments of the invention, the VEGFR/VEGF inhibitor administered in the method is cabozantinib or axitinib. In some specific embodiments of the invention, the VEGFR/VEGF inhibitor administered in the method is cabozantinib, e.g. cabozantinib (S)-malate. In other specific embodiments of the invention, the VEGFR/VEGF inhibitor administered in the method is axitinib.

In the method according to the invention, the cancer may be e.g. a cancer for which the VEGFR/VEGF inhibitor is a standard-of-care treatment option (e.g. a cancer for which cabozantinib or axitinib is a standard-of-care treatment option) and/or a cancer susceptible to anti-angiogenic treatments (based on high vascularization). In some embodiments, the cancer is selected from a group consisting of renal-cell carcinoma (RCC), colorectal cancer, lung cancer (e.g. non-small cell lung cancer), head and neck cancer, gastric cancer (e.g. gastric carcinoma), gastro-esophageal junction (GEJ) adenocarcinoma, gastrointestinal stromal tumors, glioblastoma, hepatocellular carcinoma, breast cancer, thyroid cancer, soft tissue sarcoma, chronic myeloid leukemia (CML), and Philadelphia chromosome-positive (Ph+) acute lymphoblastic leukemia (ALL). In some embodiments, the cancer is RCC. In some specific embodiments, the cancer is clear cell RCC.

In some embodiments, present invention thus relates to a method for prophylaxis and/or treatment of cancer, the method comprising administering to a subject the following compounds a) and b): a) Compound A or a physiologically acceptable salt thereof; b) cabozantinib or a physiologically acceptable salt thereof.

In some embodiments, present invention relates to a method for prophylaxis and/or treatment of RCC (such as e.g. clear cell RCC), the method comprising administering to a subject the following compounds a) and b): a) Compound A or a physiologically acceptable salt thereof; b) cabozantinib or a physiologically acceptable salt thereof.

In some embodiments, present invention relates to a method for prophylaxis and/or treatment of cancer, the method comprising administering to a subject the following compounds a) and b): a) Compound A or a physiologically acceptable salt thereof; b) cabozantinib (S)-malate.

In some embodiments, present invention relates to a method for prophylaxis and/or treatment of RCC (such as e.g. clear cell RCC), the method comprising administering to a subject the following compounds a) and b): a) Compound A or a physiologically acceptable salt thereof; b) cabozantinib (S)-malate.

In some embodiments, present invention relates to a method for prophylaxis and/or treatment of cancer, the method comprising administering to a subject the following compounds a) and b): a) Compound A or a physiologically acceptable salt thereof; b) axitinib or a physiologically acceptable salt thereof.

In some embodiments, present invention relates to a method for prophylaxis and/or treatment of RCC (such as e.g. clear cell RCC), the method comprising administering to a subject the following compounds a) and b): a) Compound A or a physiologically acceptable salt thereof; b) axitinib or a physiologically acceptable salt thereof.

In the methods according to the invention, compound A and the VEGFR/VEGF inhibitor (e.g. cabozantinib or axitinib), or their physiologically acceptable salts, can be administered via any desired suitable route, for example by oral (including buccal or sublingual), rectal, nasal, topical (including buccal, sublingual or transdermal), vaginal or parenteral (including subcutaneous, intramuscular, intravenous or intradermal) administration.

In the methods according to the invention, compound A and the VEGFR/VEGF inhibitor (e.g. cabozantinib or axitinib), or their physiologically acceptable salts, can be administered simultaneously or sequentially. When administered simultaneously, Compound A and the VEGFR/VEGF inhibitor (or their physiologically acceptable salts) may be administered as a compound mixture in one pharmaceutical composition or as separate pharmaceutical compositions.

In one embodiment, the method for prophylaxis or treatment of cancer according to the invention comprises sequential administration of Compound A and the VEGFR/VEGF inhibitor (e.g. cabozantinib or axitinib) or their physiologically acceptable salts.

In some embodiments of the method of the invention, Compound A and cabozantinib are administered once daily (i.e. QD) at a dose of 60 mg cabozantinib (e.g. cabozantinib (S)-malate) and 20 mg or 35 mg Compound A. In some embodiments, the daily administration of Compound A and cabozantinib is an oral administration. In some embodiments, the daily administration of Compound A and cabozantinib is performed in 21-day cycles.

In other embodiments of the method of the invention, Compound A and axitinib are administered at a dose of 5 mg axitinib taken twice daily (preferably 12 hours apart) and 20 mg or 35 mg Compound A taken once daily. In some embodiments, the administration of Compound A and axitinib daily and twice daily, respectively, is an oral administration. In some embodiments, the administration of Compound A and axitinib daily and twice daily, respectively, is performed in 21-day cycles.

In embodiments of the present invention, the subject to which the compounds are administered may be any mammal; in preferred embodiments, the subject is a human subject.

The present invention also relates to Compound A and a VEGFR/VEGF inhibitor (or their physiologically acceptable salts) for use in the method of prophylaxis or treatment of cancer according to the invention. In some embodiments of the invention, the VEGFR/VEGF inhibitor for such use is selected from the group consisting of aflibercept, apatinib, axitinib, bevacizumab, brivanib alaninate, cabozantinib, cediranib, lenvatinib, linifanib, pazopanib, ponatinib, ramucirumab, regorafenib, sorafenib, sunitinib, vandetanib, and 33C3. In some embodiments of the invention, the VEGFR/VEGF inhibitor for such use is cabozantinib or axitinib. In some specific embodiments, the VEGFR/VEGF inhibitor for such use is cabozantinib, e.g. cabozantinib (S)-malate. In other specific embodiments, the VEGFR/VEGF inhibitor for such use is axitinib.

The present invention also relates to the compound mixtures or pharmaceutical compositions according to the invention for use in the method of prophylaxis or treatment of cancer according to the invention.

Furthermore, the present invention relates to the use of said compound mixtures or pharmaceutical compositions for the preparation of a medicament for prophylaxis or treatment of cancer.

The expression “effective amount” denotes the amount of a medicament or of an active pharmaceutical ingredient which causes in a tissue, system, animal or human a biological or medical response which is sought or desired, for example, by a researcher or physician.

In addition, the expression “therapeutically effective amount” denotes an amount which, compared with a corresponding subject who has not received this amount, has the following consequence: improved treatment, healing, prevention or elimination of a disease, syndrome, condition, complaint or disorder, or prevention of side effects or also reduction in the progress of a disease, condition or disorder. The term “therapeutically effective amount” also encompasses the amounts which are effective for increasing normal physiological function.

A therapeutically effective amount of a compound or compound mixture of the present invention depends on a number of factors, including, for example, the age and weight of the recipient, the precise condition that requires treatment, and its severity, the nature of the formulation and the method of administration, and is ultimately determined by the treating doctor or vet. For instance, an effective amount of an API for the treatment of the diseases according to the invention can be in the range from 0.1 to 100 mg/kg of body weight of the recipient (mammal) per day, e.g. in the range from 1 to 10 mg/kg of body weight per day. Thus, the actual amount per day for an adult mammal weighing 70 kg can be in the range from 7 to 7000 mg, e.g. between 70 and 700 mg, where this amount can be administered as an individual dose per day or more usually in a series of part-doses (such as, for example, two, three, four, five or six) per day, so that the total daily dose is the same. An effective amount of a salt or of a physiologically functional derivative thereof can be determined as a fraction of the effective amount of the compounds and compound mixtures according to the invention per se.

### EXAMPLES

The following examples A1 to A8 relate to pharmaceutical compositions:

### Example A1: Injection Vials

A solution of 100 g of a compound or a compound mixture according to the invention and 5 g of disodium hydrogenphosphate in 3 l of bidistilled water is adjusted to pH 6.5 using 2 N hydrochloric acid, sterile filtered, transferred into injection vials, lyophilised and sealed under sterile conditions. Each injection vial contains 5 mg of active ingredients.

### Example A2: Suppositories

20 g of a compound or a compound mixture according to the invention is melted with 100 g of soya lecithin and 1400 g of cocoa butter, poured into moulds and allowed to cool. Each suppository contains 20 mg of active ingredients.

### Example A3: Solution

A solution is prepared from 1 g of a compound or a compound mixture according to the invention, 9.38 g of NaH₂PO₄ × 2 H₂O, 28.48 g of NaH₂PO₄ × 12 H₂O and 0.1 g of benzalkonium chloride in 940 ml of bidistilled water. The pH is adjusted to 6.8, and the solution is made up to 1 l and sterilised by irradiation. This solution can be used in the form of eye drops.

### Example A4: Ointment

500 mg of a compound or a compound mixture according to the invention are mixed with 99.5 g of Vaseline under aseptic conditions.

### Example A5: Tablets

1 kg of a compound or a compound mixture according to the invention (e.g. Compound A), 4 kg of lactose, 1.2 kg of potato starch, 0.2 kg of talc and 0.1 kg of magnesium stearate is pressed to give tablets in a conventional manner in such a way that each tablet contains 10 mg of active ingredients.

### Example A6: Coated Tablets

Tablets are pressed analogously to Example A5 and subsequently coated in a conventional manner with a coating of sucrose, potato starch, talc, tragacanth and dye.

### Example A7: Capsules

2 kg of a compound or a compound mixture according to the invention (e.g. Compound A) are introduced into hard gelatine capsules in a conventional manner in such a way that each capsule contains 20 mg of the active ingredient(s). Alternatively, for example, Compound A is introduced into hard gelatin capsules in a conventional manner in such a way that each capsule contains 1 mg, 5 mg or 30 mg of the active ingredient.

### Example A8: Ampoules

A solution of 1 kg of a compound or a compound mixture according to the invention in 60 l of bidistilled water is transferred into ampoules, lyophilised under aseptic conditions and sealed under sterile conditions. Each ampoule contains 10 mg of active ingredients.

The following examples B1 and B2 relate to combination studies using Compound A and VEGFR/VEGF inhibitors. It has been found by the inventors of the present patent application that Compound A achieves a significant combination benefit when combined with cabozantinib or axitinib in the treatment of a cancer, as reported in more detail below:

### Example B1: In Vivo Efficacy of Compound A in Combination With Axitinib or Cabozantinib in RCC Patient Derived Tumor Models (PDX)

### 1. Summary

This example characterizes the antitumor activity of Compound A in combination with axitinib or cabozantinib (SoC for RCC) in sixteen RCC patient derived tumor xenograft models (PDX). While Compound A, axitinib and cabozantinib treated tumors progressed under monotherapy treatment, combination of both agents (i.e. combination of Compound A with axitinib or combination of Compound A with cabozantinib) showed a significant combination benefit resulting in an increase in progression-free survival as well as overall survival compared to monotherapy. Treatments were well tolerated.

### 2. List of Abbreviations

| | |
|---|---|
| aai | Aqua ad iniectabilia, water for injection |
| AC | Air change |
| BID | Twice weekly |
| BW | Body weight |
| BWL | Body weight loss |
| CMC | Carboxymethyl cellulose |
| D5W | 5% dextrose in water |
| EDTA | Ethylene diamine tetra acetic acid |
| ELN | Electronic labor notebook |
| H:0 | First dose of the day = h:0 |
| H:0+12 | Dosing twice daily, 12 hours apart |
| IT | Induction time |
| IVC | Individually ventilated cages |
| kg | Kilogram |
| MetAP2 | Methionine amino peptidase 2 |
| mg | Milligram |
| ml | Milliliter |
| MTD | Maximum tolerated dose |
| n/a | Not applicable |
| NMRI | Naval Medical Research Institute |
| NMRI Foxn1nu | Athymic immunodeficient mutant on NMRI background |
| n.r. | Not reached |
| PBS | Phosphate-buffered saline |
| PDX | Patient-derived xenograft |
| p.o. | Per os, oral(ly) |
| QD*n | Once daily for n days |
| RXA | Renal xenograft of Asian ethnicity |
| RXF | Renal xenograft Freiburg |
| RTV | Relative tumor volume |
| ROP | Research operating procedure |
| SOC | Standard of care |
| SOP | Standard operating procedure |
| T/C | Test versus control value |
| TV | Tumor volume |
| vs. | Versus |

### 3. Materials and Methods

### 3.1 Materials

### 3.1.1 Test Systems

### 3.1.2 Test Material and Positive Control Material

### 3.1.2.1 Animals

All experiments and protocols were approved by animal welfare body and local authorities, and were conducted according to all applicable international, national and local laws and guidelines. Only animals with unobjectionable health were selected to enter testing procedures. The animals (female NMRI nu/nu mice (NMRI-Foxn1^{nu}); Envigo RMS GmbH, Netherlands) were delivered at the age of four to six weeks and were used for experiments after at least one week of acclimatization. Animals were arbitrarily numbered during tumor implantation using radio frequency identification transponders. Each cage was labeled with a record card indicating all relevant experimental details.

### 3.1.2.2 Housing Conditions

Animals were housed in individually ventilated cages (TECNIPLAST Sealsafe™-IVC-System, TECNIPLAST, Hohenpeissenberg, Germany), depending on group size, either in type III or type II long cages. They were kept under a 14L:10D artificial light cycle. The temperature inside the cages was maintained at 25 ± 1° C. with a relative humidity of 40-70% and 60-65 air changes per hour in the cage. Dust-free bedding consisting of aspen wood chips with approximate dimensions of 5 × 5 × 1 mm (ABEDD® - LAB & VET Service GmbH, Vienna, Austria, Product Code: LTE E-001) and additional nesting material were used. The cages including the bedding and the nesting material were changed weekly. The animals were fed autoclaved Teklad Global 19% Protein Extruded Diet (T.2019S.12) from Envigo RMS SARL and had access to sterile filtered and acidified (pH 2.5) tap water that was changed twice weekly. Feed and water were provided ad libitum. All materials were autoclaved prior to use. Where necessary, animals were provided with a nutrient fortified water gel (DietGel® Recovery from Clear H₂O, Maine, USA), which was changed every other day.

### 3.1.2.3 Test Tumors

The tumor xenografts were derived from surgical specimens from human cancer patients. Following excision at surgery, tumor pieces were subcutaneously implanted into immunodeficient mice and are therefore referred to as patient-derived tumor xenografts (PDX). Establishment and characterization of the PDXs was performed following their primary implantation into immunodeficient mice (passage 1). The tumor xenografts were passaged until establishment of a stable growth pattern. At that point, master stocks of early passage PDXs were frozen in liquid nitrogen. Further information on each PDX (e.g. histology, growth characteristics) is presented in Table 1.

TABLE 1

Characteristics of 16 RCC Patient Derived Xenografts

| | | | |
|---|---|---|---|
| Tumor Designation | Histology/Differentiation | Orgin | Stage¹ |
| RXA SMTCA75 | Not known / not known | Not known | |
| RXF 1220 | Clear cell carcinoma / not known | Metastasis | T3M1 lung |
| RXF 2178 | Clear cell carcinoma / not known | Primary | pT4, pN2 (9/10), pM1 R1 |
| RXF 2264 | Clear cell carcinoma / not known | Not known | Not available |
| RXF 2282 | Clear cell carcinoma / not known | Not known | TNM pT3b,N0;G3 |
| RXF 2502 | Clear cell carcinoma / not known | Not known | |
| RXF 2503 | Clear cell carcinoma / not known | Not known | |
| RXF 2519 | Clear cell carcinoma / not known | Primary | |
| RXF 2521 | Clear cell carcinoma / not known | Not known | |
| RXF 2717 | Clear cell carcinoma / not known | Not known | pT3a, pNx, V1, G3-4 Ro |
| RXF 2720 | Clear cell carcinoma / not known | Not known | TpT3b,pNo(0/6), cMo, V1, G2, lokal R1 |
| RXF 2755 | Clear cell carcinoma / not known | Not known | Not available |
| RXF 2796 | Clear cell carcinoma / not known | Not known | Not available |
| RXF 2827 | Not known / not known | Not known | Not available |
| RXF 2829 | Clear cell carcinoma / not known | Not known | Not available |
| RXF MRI-H-166 | Not available / not known | Not known | Not available |

¹ Stage according to the TNM Staging Guide or according to the International Classification of Diseases for Oncology (ICD-O, issued by the World Health Organization)

TABLE 2

Experimental Overview

| Tumor Designation/Passage¹ | Exp. No. | Number(Gender of Animals | Tumors implanted per Animal | Number of Randomized Animals | GRoup Mean Tumor Volume² |
|---|---|---|---|---|---|
| RXA SMTCA75 / 11N3 | T209 | 58 / female | 1 (40) / 2 (18) | 30 | 108.0-108.8 |
| RXF 1220/ 11N4 | T199 | 43 / female | 1 | 30 | 114.8-116.3 |
| RXF 2178 / 11N4 | T201 | 43 / female | 1 | 30 | 133.3-135.9 |
| RXF 2264 / 7N3 | T202 | 55 / female | 1 | 30 | 116.9-118.2 |
| RXF 2282 / 18N11 | T197 | 47 / female | 1 | 30 | 105.8-108.7 |
| RXF 2502 / 13N6 | T208 | 43 / female | 1 | 18 | 91.9-97.7 |
| RXF 2503 / 14N5 | T206 | 50 / female | 1 | 30 | 108.1-109.9 |
| RXF 2519 / 11N3 | T204 | 50 / female | 1 | 30 | 112.5-116.2 |
| RXF 2521 / 12N4 | T210 | 50 / female | 1 | 30 | 102.1-103.2 |
| RXF 2717 / 19N9 | T198 | 60 / female | 1 | 21 | 75.8-106.0 |
| RXF 2720 / 11N3 | T205 | 50 / female | 1 | 30 | 108.1-108.6 |
| RXF 2755 / 16N4 | T200 | 47 / female | 1 | 30 | 77.0-80.3 |
| RXF 2796 / 8N3 | T207 | 50 / female | 1 | 30 | 107.0-108.9 |
| RXF 2827 / 5N2 | T212 | 50 / female | 1 | 30 | 114.8-116.4 |
| RXF 2829 / 5N3 | T211 | 50 / female | 1 | 30 | 110.6-111.7 |
| RXF MRI-H-166 / 16N4 | T203 | 43 / female | 1 | 30 | 112.5-114.3 |

¹ The number preceding the N represents the total number of passages and the number following the N represents the passage number after the last freeze-thaw cycle. ² Range at randomization [mm³].
* Due to the low take rate in the RXA SMTCA75 model, 18 animals were implanted bilaterally and 40 animals unilaterally. Before enrollment one tumor was explanted in mice bearing two tumors.

### 3.1.3 Test Material

| Name | Batch | Supplier |
|---|---|---|
| Axitinib (free base) | AXB-106 | LC Laboratories |
| Cabozantinib S-malate | 18416 | Medchem Express |
| Compound A (free base) | 11 | Merck KGaA |

Vehicle for axitinib: 0.5% carboxymethyl cellulose (CMC; used also as control vehicle 1).

Vehicle for cabozantinib: 30% propylene glycol, 5% Tween 80, 65% D5W (wherein D5W means 5% dextrose in water).

Vehicle for Compound A: 0.25% Methocel (Colorcon) in sterile water for injection (wfi) (sterile wfi; used also as control vehicle 2).

Axitinib: A dosing solution of 0.4 mg/ml for dosing at 4 mg/kg/dose was prepared daily by dissolving 6.4 mg dry matter in 16 ml vehicle and stirring for at least 30 min. If necessary, the solution was sonicated for 2 min.

Cabozantinib: A dosing solution of 1 mg/ml for dosing at 10 mg/kg/dose was prepared directly before administration by slowly adding 0.2 ml 100% propylene glycol to 12 mg cabozantinib. It was stirred vigorously on a magnetic stirrer until a fine suspension with clumps formed, followed by vortexing briefly for 15-20 s. A further 3.4 ml propylene glycol was added and the stirring and vortexing steps were repeated. Then 600 µl 100% Tween 80 was added while stirring continuously followed by vortexing briefly for 15-20 s. The solution was then sonicated for 10 min and 7.8 ml D5W was added while stirring slowly. At this stage the solution was clear with lots of air bubbles. Care was taken to exclude these when drawing the suspension into a syringe for administration of therapy.

Compound A: A dosing solution of 10 mg/ml for dosing at 100 mg/kg/dose was prepared every three days by dissolving 180 mg dry matter in 18 ml vehicle. The resulting white suspension was stirred for two hours on a magnetic stirrer with a hot plate (Phoenix Instruments, model RSM-10HS) at 50° C. The suspension was brought to room temperature and mixed well each time before transferring to a syringe for dosing.

All therapy solutions were administered in a dose volume of 10 ml/kg.

### 3.1.4 Solutions

All reagents and buffers were stored according to the instructions of the manufacturers and used before the batch expiration date. Compound A was developed at Merck KGaA, Darmstadt, Germany.

| | |
|---|---|
| Abbreviated name | Origin and contents |
| Methocel | Methocel K4M Premium (Hypromellose 2208 from Colorcon) |

The vehicle for Compound A, 0.25% Methocel® in aai, was made by dissolving 875 mg Methocel® in 200 ml water for injection and stirring at 1000 rpm at 65.5° C. for six hours until dissolved. Then 150 ml water was added, and the solution was stirred for a further 5 minutes before sterile filtration.

### 3.1.5 Instruments

| | |
|---|---|
| Instrument | Supplier |
| Computer: Dell Optiplex version 3010 | Dell |
| Caliper: Sender: Sylvac S_Cal PRO Receiver: IBRit-rf1 usb Software: IBR SimKey Version 29.01.2010 | Sender: Sylvac, Switzerland (Studenroth) Receiver: IBR Messtechnik GmbH & Co. KG Software: IBR Messtechnik GmbH & Co. KG |
| Scale: Kern PCB Software: Winwedge | Kern Software: TAL Technologies Inc., USA |
| RFID reader: Planet ID “Tatjana”-reader, Pyramid receiver Software: Pyramid Server 3.1.1, Planet routing 4.5.4. | Reader: Planet ID Software: Planet ID |
| Magnetic stirrer | Phoenix Instruments, RSM-10HS |

### 3.2 Doses in Test

### 3.2.1 Test Material Concentrations

| Test Article | Dose in mg/kg | Dose in ml/kg | Route of administration | Treatment schedule |
|---|---|---|---|---|
| Compound A | 100 | 10 | p.o. | QD |
| axitinib | 4 | 10 | p.o. | BID |
| cabozantinib | 10 | 10 | p.o. | QD |

### 3.2.2 Negative Controls

| Negative Control | Dose in mg/kg | Dose in ml/kg | Route of administration | Treatment schedule |
|---|---|---|---|---|
| 0.25% Methocel in aai | / | 10 | p.o. | QD |
| 0.5% CMC low viscosity | / | 10 | p.o. | BID |

### 3.3 Study Procedure

Tumor cell inoculation: Tumor fragments were obtained from xenografts in serial passage in nude mice. After removal from donor mice, tumors were cut into fragments (3-4 mm edge length) and placed in PBS containing 10% penicillin/streptomycin. Recipient animals were anesthetized by inhalation of isoflurane and received unilateral or bilateral tumor implants subcutaneously in the flank. Tumor xenografts with a take rate < 65% were implanted with one or two tumors per mouse and in case of a bilateral take, one of these tumors was explanted prior to randomization.

Randomization: Animals and tumor implants were monitored daily until solid tumor growth was detectable in a sufficient number of animals. At randomization, the volume of growing tumors was determined. Animals fulfilling the randomization criteria (i.e. bearing tumors of 50-250 mm³, preferably 80-200 mm³) were then distributed into experimental groups, aiming at comparable median and mean group tumor volumes. Animals not used for experiments where available were used as satellite animals for PD sampling. The day of randomization was designated as day 0 of the experiment. The time from implantation to randomization at a standard tumor volume is expressed in days as “Induction time (IT)”. Induction times of tumors are routinely recorded, and a median IT is calculated for characterization purposes.

Tumor measurements and calculations: Tumor length (L) and width (W) were measured twice weekly by calipers. The tumor volume was calculated using the formula L×W²/2. Relative volumes of individual tumors (individual RTVs) for Day x were calculated by dividing the absolute individual tumor volume on Day x (Tₓ) by the absolute individual tumor volume of the same tumor on the day of randomization (Tᵣ) multiplied by 100%.

Body weight: Animals were weighed twice a week, or daily if body weight loss in excess of 10% was recorded. Relative body weights of individual animals were calculated by dividing the individual body weight on Day X (BWₓ) by the individual body weight on the day of randomization (BWᵣ) multiplied by 100%. Group mean relative body weights were calculated as well for evaluation purposes.

Euthanasia criteria: According to animal welfare regulations and the relevant SOP, the following euthanasia criteria apply to individual animals, irrespective of the experimental status:
tumor volume > 2000 mm³ (unilateral)
ulcerating, skin-penetrating tumor
body weight loss > 30% on one measuring day
continued body weight loss > 20% for more than two days
recorded, rapid decrease in body weight > 20% within two days
severe impairment of general condition (apathy, pain, markedly reduced feed and water intake, dyspnea, abnormal habitus or behavior)

Where individual animals fulfilled euthanasia criteria, sampling was performed ahead of the scheduled time and, if feasible, at the correct time interval after administration of the last applicable dose.

Sampling: Samples were collected according to all relevant animal welfare guidelines and under sterile conditions. No samples were taken from animals that were found dead. Tumors were collected immediately after euthanasia, and divided into two parts such that one part was approx. 100 mg. This weight of this fragment was documented before it was placed in a tubes (Precellys) and transferred to liquid nitrogen for snap freezing. The remaining tumor piece was fixed in formalin. The fixation was performed in 10% neutral phosphate-buffered formalin for approximately 24 hours. The fixative was then replaced by submerging the samples in PBS and samples were directly infiltrated by paraffin for embedding. If tumor material was limited, priority was given to FFPE samples.

Blood was collected by cardiac puncture under isoflurane anesthesia (terminal collection of mixed venous and arterial blood). Serum was prepared by incubating the blood in standard serum vials for 30 minutes at room temperature followed by centrifugation at 10000×g for 5 minutes. Serum samples were stored at -80° C.

### 3.4 Endpoints

Preclinical response criteria have been defined following Therasse et al., J Nat Cancer Inst 2000; 92(3):205-16.

Tumor progression: tumor progression was defined as treatment groups or individual mice reaching a tumor volume change at the end of the experiment or treatment of 73% compared to the tumor volume at the start of treatment (where tumor volume change at the start was 0%). (In other words: If relative tumor volume (RTV) was set as 100% at the start, then tumor progression was defined as treatment groups reaching a median tumor volume of 173%).

Tumor stasis: Tumor volume change between -66% and 73% by the end of treatment

Tumor regression: Tumor volume change ≤ -66% by the end of treatment

Progression free survival: Prolonged treatment phase in all experiments up to 70 days. Progression free survival has been defined when tumors have reached a relative tumor volume (RTV) of 173% compared to RTV at start of treatment (= 100%).

Overall survival: Prolonged treatment phase in all experiments up to 70 days. Overall survival has been defined when tumors have reached a relative tumor volume (RTV) of 1000% compared to RTV at start of treatment (= 100%).

MTD in repeat dose studies: The maximum tolerated dose (MTD) is the dose or exposure that causes no mortality, body weight loss of < 20% and no irreversible clinical or pathological findings. A dosage resulting in 20% body weight change (mean of group) or ≥ 10% drug related deaths was considered as toxic dosage. Animal body weights included the tumor weights.

TABLE 3

Groups and treatments

| Group ID | Therapy¹ | Dose Level [mg/kg/day] | Schedule [Day] | Route |
|---|---|---|---|---|
| 1 | Control Vehicle 1// Control Vehicle 2 | 10/2*10/10 ml/kg// 10 ml/kg | 0(h:12)/1-44(h:0+12)/45(h:0+6)/46-52(h:0+12)/53(h:0+6)/54-69(h:0+12)/70(h:0)// 1-70(h:2) | p.o.// p.o. |
| 2 | Control Vehicle 1// Compound A | 10/2*10/10 ml/kg// 100 | 0(h:12)/1-44(h:0+12)/45(h:0+6)/46-52(h:0+12)/53(h:0+6)/54-69(h:0+12)/70(h:0)// 1-70(h:2) | p.o.// p.o. |
| 3 | Control Vehicle 2 //Cabozantinib | 10 ml/kg// 10 | 1-70(h:2)// 1-70(h:0) | p.o.// p.o. |
| 4 | Control Vehicle 2 //Axitinib | 10 ml/kg// 4/2*4/4 | 1-70(h:2)// 0(h:12)/1-44(h:0+12)/45(h:0+6)/46-52(h:0+12)/53(h:0+6)/54-69(h:0+12)/70(h:0) | p.o.// p.o. |
| 5 | Compound A// Cabozantinib | 100// 10 | 1-70(h:2)// 1-70(h:0) | p.o.// p.o. |
| 6 | Compound A// Axitinib | 100// 4/2*4/4 | 1-70(h:2)// 0(h:12)/1-44(h:0+12)/45(h:0+6)/46-52(h:0+12)/53(h:0+6)/54-69(h:0+12)/70(h:0) | p.o.// p.o. |

¹ Vehicle for Compound A and control vehicle 2: 0.25% Methocel in sterile wfi; vehicle for axitinib and control vehicle 1: 0.5% CMC low viscosity; vehicle for cabozantinib: 30% propylene glycol, 5% Tween 80, 65% D5W (5% dextrose in water).

### 3.5 Computer Programs Used and Statistical Evaluation

| | |
|---|---|
| Program | Version |
| GraphPad Prism (Visualization of body weight changes) | 5.04 |
| R Bioconductor (Visualization of survival time and statistical evaluation) | R-3.6.1, packages ggplot2, survival, survminer |

Statistical analysis: Statistical analysis of Progression Free Survival (PFS) and Overall Survival (OS) time has been performed via Log-rank (Mantel-cox) test.

### 4. Results

The anti-tumor activity of Compound A in combination with SoC agents axitinib or cabozantinib was evaluated in 16 selected subcutaneous patient derived xenograft models of RCC. Tumor fragments from the patient derived tumor types were grown in donor mice, harvested and further implantation in mice. In each efficacy study, tumor-bearing animals were assigned to experimental groups (n = 5) having the same average tumor size at the start of treatment, which was designated as Day 0. Compound A was orally administered QD with a dose of 100 mg/kg for a defined maximum time period of 70 days. Axitinib and cabozantinib were administered with a dose of 4 mg/kg (BID) and 10 mg/kg (QD), respectively. Same doses and schedules were applied for the combination treatment. Tumors and animal weights were measured twice weekly; tumor volume (mm³) was calculated with the equation [length × width²/2]. Tolerability was assessed based on percent body weight difference during the treatment period. Progression free survival (PFS) and overall survival (OS) time has been assessed based on defined endpoints (PFS= relative tumor volume (RTV) 173%, OS= RTV >1000%) over the whole treatment period of 70 days.

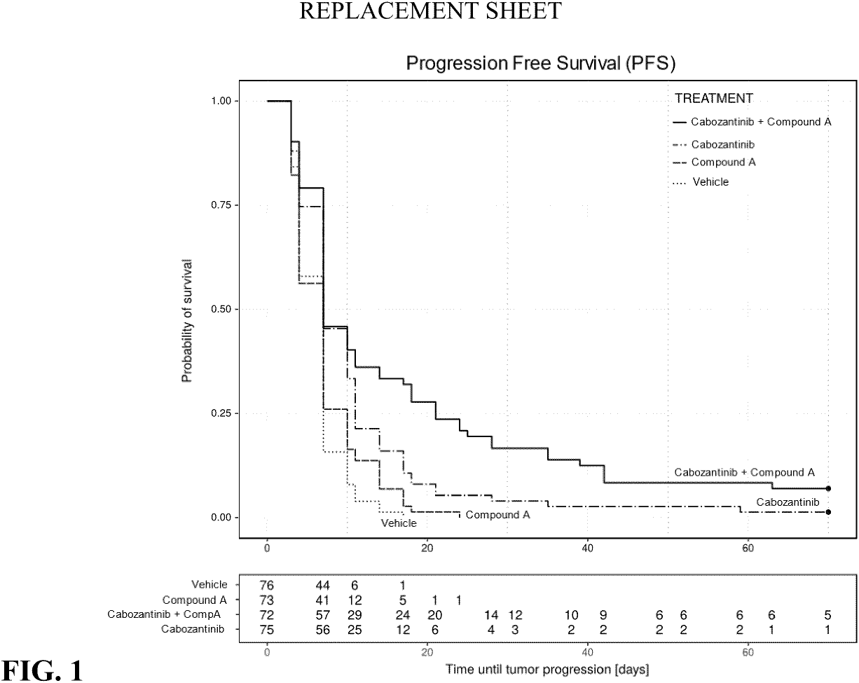

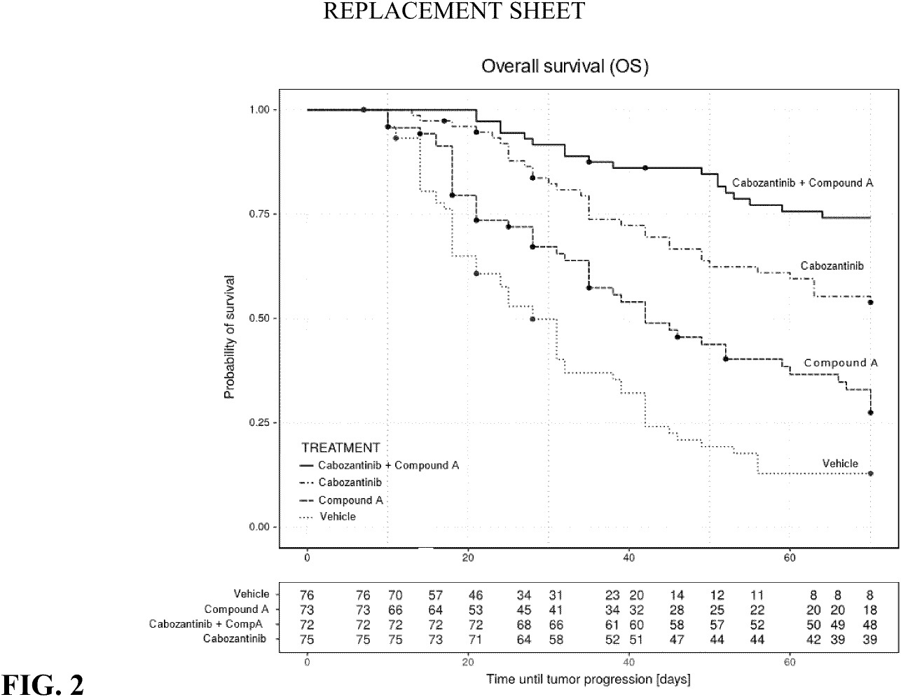

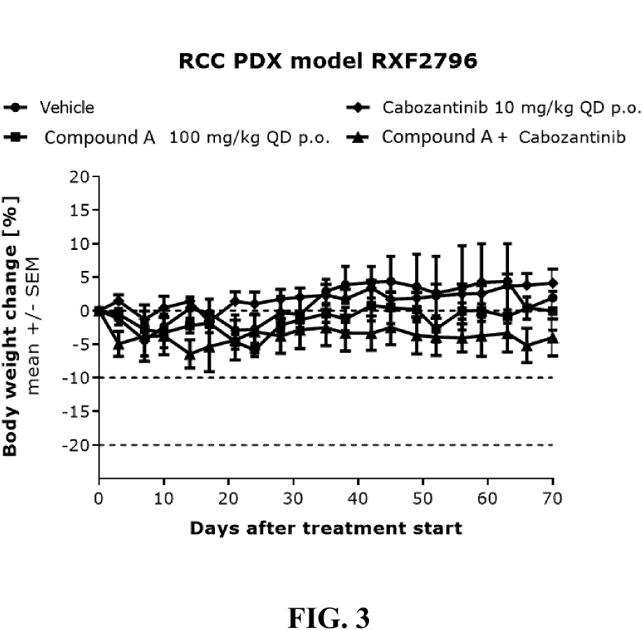

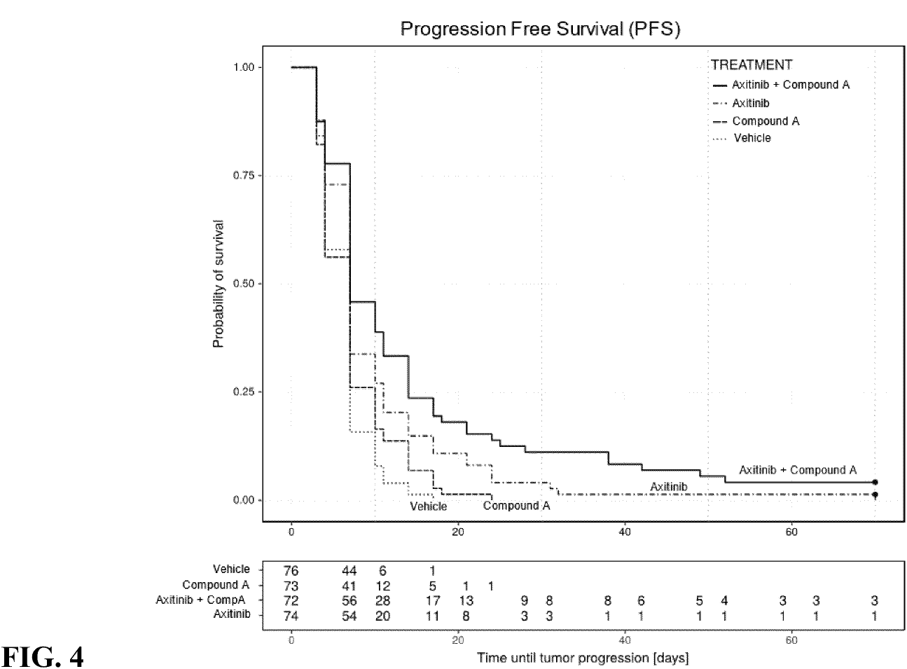

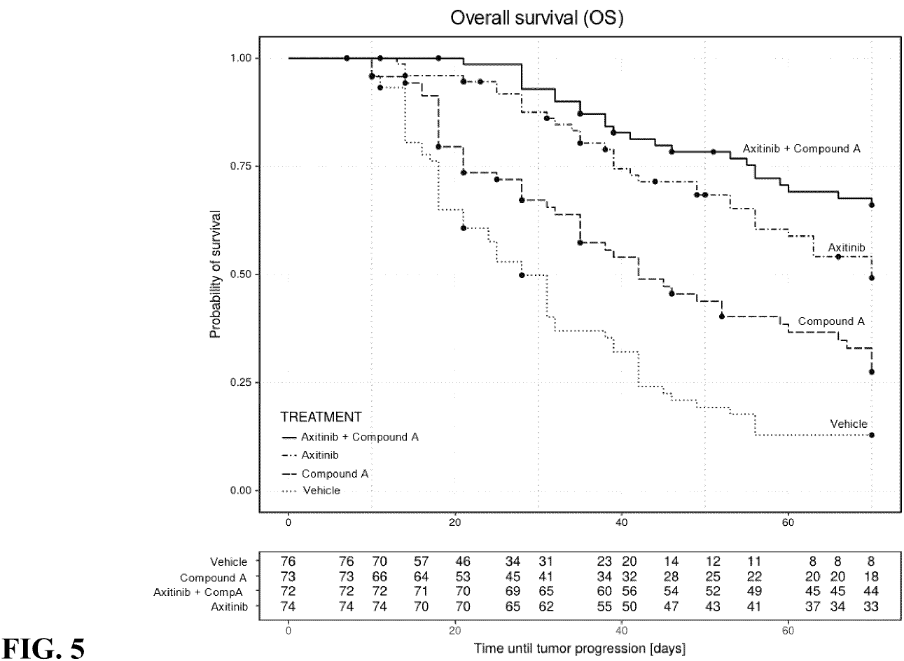

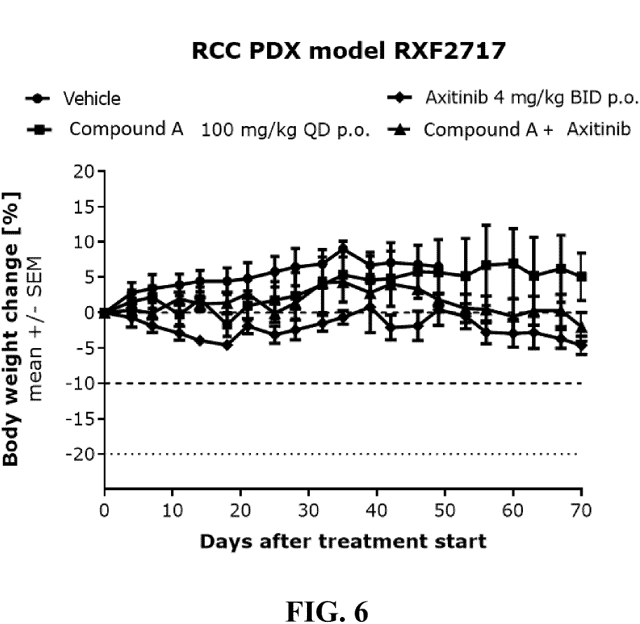

Overall, Compound A, axitinib and cabozantinib monotherapies did not strongly inhibit tumor growth, and tumors progressed under treatment. Combination of Compound A with axitinib or cabozantinib showed a significant combination benefit resulting in an increase in progression-free survival (FIGS. 1 and 4) and overall survival (FIGS. 2 and 5). All treatments were well tolerated (see body weights: one representative example of a PDX model shown per treatment group, see FIGS. 3 and 6). RECIST like preclinical response have been assessed following the definitions in Therasse et al. 2000.

### Example B2: An Open-Label, Phase I, Dose Escalation Trial of Methionine Aminopeptidase 2 Inhibitor Compound a in Subjects With Advanced Solid Tumors

| Title | An Open-label, Phase I, Dose Escalation Trial of Methionine Aminopeptidase 2 Inhibitor Compound A in Subjects with Advanced Solid Tumors |
|---|---|
| Trial Phase | I/Ib |

Methodology:
This is a 2-part, Phase I/Ib, first in human, multicenter, open-label trial of Compound A administered daily.
Part 1 is a dose-escalation study designed to explore the safety, tolerability, PK and Pd profiles, and clinical activity of Compound A as a single agent across cycles of 21-day treatment, in subjects with advanced solid tumors with no surgical, radiation, or systemic anticancer therapies available.
Part 2 is designed to determine the safety, tolerability, and anti-tumor activity of Compound A combined with cabozantinib in subjects with mRCC. In Part 2A, a dose escalation (Part 2A) and an expansion cohort (Part 2B) design are planned.
In Part 2A, escalating doses of Compound A will be combined with cabozantinib in subjects with mRCC who have progressed to 1 or more previous lines of systemic anticancer therapy, excluding treatment with cabozantinib. The Part 2A of the study allows subjects accrual in a staggered manner. Thus, enrollment in Part 2A can occur while Part 1 is still ongoing; however, enrollment will only be at a dose level of Compound A that has already been determined safe in Part 1 of the study. An exploratory food effect assessment will be performed on Day -1 of Cycle 1 in which subjects will receive a single dose of Compound A as single agent with food.
In the expansion Part 2B, Compound A at the recommended dose for expansion (RDE) / maximum tolerated dose (MTD) defined in Part 2A will be combined with cabozantinib in subjects with mRCC who have progressed to 1 or 2 previous lines of systemic anticancer therapy, excluding treatment with cabozantinib. In Part 2B, subjects should have failed to only 1 previous antiangiogenic tyrosine kinase inhibitor (TKI) for metastatic disease. Adjuvant therapy with sunitinib will be considered as 1 line of therapy for metastatic disease in the case that disease progression occurs during or within 3 months of the completion of the treatment.

Planned number of subjects: Part 1: 36-42 subjects. Part 2A: 6-15 subjects. Part 2B: 30-40 subjects.
Part 1, Compound A Single Agent, Solid Tumors
Primary endpoints:
• Dose limiting toxicities (DLTs) during the first 21-day treatment cycle, based on a pre-defined set of adverse events (AEs), to determine the MTD.
Secondary endpoints:
• Occurrence and severity of treatment-emergent adverse events (TEAEs), and deaths, including cause of death, from screening up to the End of Treatment visit (EOT)
• Changes in clinical laboratory measures, electrocardiogram (ECG) measures, vital signs, Eastern Cooperative Oncology Group Performance status (ECOGPS)
• Best overall response (BOR: complete response [CR], partial response [PR], stable disease [SD] or progressive disease [PD]) according to RECIST v1.1. criteria as assessed by Investigators
• Clinical benefit defined as CR, PR, and SD for ≥ 12 weeks
• PFS (progression-free survival) time.
Part 2, Compound A Combined with Cabozantinib, mRCC
Part 2A, Dose Escalation
Primary endpoints:
• Occurrence of DLTs in subjects with mRCC receiving Compound A combined with cabozantinib during the first 22-day treatment cycle (21 days of Compound A combined with cabozantinib, preceded by 1 day of Compound A alone) based on predefined set of AEs
• Occurrence of TEAEs (including TEAEs leading to death or discontinuation of treatment) in subjects with mRCC receiving Compound A combined with cabozantinib. Any AEs that occur or worsen between start of study intervention and 30 days after end of study intervention will be considered TEAEs
• Study clinically relevant abnormal changes in clinical laboratory measures from baseline, vital signs, ECOG performance status, and ECGs from start of study intervention to 30 days after end of study intervention
• Objective Response according to RECIST v1.1 as assessed by Investigators

Secondary endpoints:
• Duration of Response (DoR) according to RECIST v1.1 criteria as assessed by Investigators
• PFS time according to RECIST v1.1 criteria as assessed by Investigators
Part 2B, Expansion Cohort
Primary endpoints:
• Occurrence of study serious AEs including deaths from first dose of study intervention to 30 days after end of study intervention
• Study clinically relevant abnormal changes in clinical laboratory measures from baseline, vital signs, ECOG PS, and ECGs from start of study intervention to 30 days after end of study intervention
• Occurrence of TEAEs (including deaths) from start of the study intervention to 30 days after end of the study intervention
• Objective response according to RECIST v1.1 criteria as assessed by Investigators
Secondary endpoints:
• DoR according to RECIST v1.1 criteria as assessed by Investigators
• PFS time according to RECIST v1.1 criteria as assessed by Investigators
Diagnosis and key inclusion and exclusion criteria:
Part 1, Compound A Single Agent, Solid Tumors
Key inclusion:
• Subjects must be refractory to or intolerant of existing cancer therapy(ies) known to provide clinical benefit
• Histologically confirmed advanced solid tumors with no clear curative treatment options available after at least 1 prior systemic anticancer therapy
• Tumor accessible for biopsies and agreement to conduct pre-dose and post-dose fresh tumor biopsies
• Male or female subjects at least 18 years of age.
Key exclusion:
• ECOG PS ≥ 2
• Extensive prior radiotherapy on more than 30% of bone marrow reserves, or prior bone marrow/stem cell transplantation within 5 years of study start
• Severe bone marrow, renal or liver impairment

Part 2, Compound A Combined with Cabozantinib, mRCC
Key inclusion:
• Histologic or cytologic evidence/proven of mRCC with clear cell component
• Previous treatments:
a. Part 2A: Subjects should have progressed to 1 or more previous lines of systemic anticancer therapy, excluding treatment with cabozantinib
b. Part 2B: Subjects should have progressed to 1 or 2 previous lines of systemic anticancer therapy, excluding treatment with cabozantinib. Subjects should have failed to only 1 previous TKI for metastatic disease. Adjuvant therapy with sunitinib will be considered as 1 line of therapy for metastatic disease in the case that disease progression occurs during or within 3 months of the completion of the treatment.
• At least 1 lesion that is measurable using RECIST v1.1.
• Adequate organ function (i.e. liver, kidney, bone marrow), as evidenced by multiple laboratory value results within specific parameters
Key exclusion:
• Previous use of cabozantinib or a MetAP2 inhibitor
• Thromboembolic events requiring therapeutic anticoagulation. Concomitant anticoagulation at therapeutic doses with oral anticoagulants (i.e., warfarin, direct thrombin and Factor Xa inhibitors) or platelet inhibitors (i.e., clopidogrel) started within the last 6 months of screening
Note: Low dose aspirin for cardio-protection and low dose low-molecular-weight heparin (LMWH) are permitted (in subjects who are on a stable dose of LMWH for at least 6 weeks before the first dose of study treatment, and who have had no clinically significant hemorrhagic complications from the anticoagulation regimen or the tumor)
• Current significant cardiac conduction abnormalities, QT corrected for heart rate according to Fridericia (QTcF) interval prolongation of > 450 ms for males and > 470 ms for females or cardiovascular disorders, including symptomatic congestive heart failure, unstable angina pectoris, and serious cardiac arrhythmias.
Investigational Medicinal Product: dose/mode of administration/ dosing schedule:
Part 1, Compound A Single Agent, Solid Tumors
Compound A in capsule form will be administered orally QD, according to the dose escalation schedule and as determined by the safety monitoring committee (SMC). The starting dose of initial dose escalation cohort will be 7 mg QD. A Bayesian two-parameter logistic regression model with overdose control will be used to assist the SMC with dose selection during the dose escalation part. The model incorporates nonclinical toxicity and observed DLT information from all previously completed dose escalation cohorts to provide a recommended dose for the next cohort.

Part 2, Compound A Combined with Cabozantinib, mRCC
Part 2A, Dose Escalation
Compound A in capsule form combined with cabozantinib will be administered orally QD in the morning in fasted conditions. Three pre-specified dose levels of Compound A are planned (20 mg, 35 mg and 60 mg) combined with 60 mg of cabozantinib.
A Bayesian two-parameter logistic regression model with overdose control will be used to assist the SMC with dose selection for the next cohort and to determine the MTD/RDE of Compound A combined with cabozantinib 60 mg QD for Part 2B. Dose levels may be added or skipped for others that are not part of the pre-specified but they will never exceed the dose level of Compound A that has already been declared to be safe in Part 1.
Cabozantinib will be used at the approved 60 mg-free-base equivalent (FBE) daily dosage with dosage adjustments to 40 mg FBE or 20 mg FBE permitted to manage AEs.
Part 2B, Dose Expansion
In Part 2B, subjects will receive Compound A at the dose level defined in the Part 2A and cabozantinib at 60 mg QD in 21-day cycles until disease progression, unacceptable toxicity, withdrawal of consent, or any criterion for withdrawal from the study.
In part 2B, the SMC will continue to monitor the safety of Compound A combined with cabozantinib and may recommend on continuation on the same dose, change in dose (not higher than MTD defined in Part 2A), or stop of the expansion cohort. Unacceptable toxicity for the expansion (Part 2B) cohort will be AEs that fulfill the DLT criteria. The SMC will evaluate the safety and cumulated toxicity of the combination therapy after 10 and after 20 subjects, who have finished their DLT period or dropped out. The SMC decisions in the expansion cohort will also be supported by a two-parameter Bayesian logistic regression model. Enrollment of subjects will not be suspended between SMCs.
Planned trial and treatment duration per subject:
Study duration per subject is approximately 8 months for Part 1, and 12 months for Part 2 including screening, treatment and follow up. Treatment is administered in consecutive 21-day cycles of continuous treatment. Subjects will receive study drug at the pre-specified dose level until disease progression, unacceptable toxicity, withdrawal of consent, or any criterion for withdrawal from the trial.
Statistical methods:
Part 1, Compound A Single Agent, Solid Tumors
Analyses will be prepared by dose level. There is no formal significance level for this trial and all analyses are considered descriptive.
Dose escalation will be aided by a Bayesian two-parameter logistic regression model. The SMC dedicated to dose escalation decisions will receive results of a Bayesian two-parameter logistic model with overdose control updated with the observed DLT data. Recommendation will be based on a loss function.
Part 2, Compound A Combined with Cabozantinib, mRCC
Part 2A, Dose escalation
A Bayesian two-parameter logistic regression model with overdose control will be used to assist the SMC to select the next dose of Compound A from 3 pre-specified dose levels (20 mg, 35 mg, and 60 mg QD). The SMC may add or skip a dose, as long as it does not exceed the dose level of Compound A that has already been declared to be safe in Part 1.
In the exploratory food effect assessment, the PK data generated for a single dose of Compound A as single agent with food will be compared with those obtained from the Part 1 of the study, in which subjects received Compound A single agent at the same dose level in fasted conditions (using a modelling approach).
Part 2B, Expansion cohort
A 2-stage design with an interim analysis for futility will be used to evaluate response.

1. A compound mixture of compounds a) and b), comprising:
a) (S)-3-Hydroxy-1-(1H-indol-5-y1)-2-oxo-pyrrolidine-3-carboxylic acid 3,5-difluoro-benzylamide or a physiologically acceptable salt thereof, and
b) an inhibitor of one or more of VEGFR1, VEGFR2, VEGFR3_{.} and VEGF, or a physiologically acceptable salt thereof.

2. The compound mixture according to claim 1, wherein the inhibitor is selected from the group consisting of aflibercept, apatinib, axitinib, bevacizumab, brivanib alaninate, cabozantinib, cediranib, lenvatinib, linifanib, pazopanib, ponatinib, ramucirumab, regorafenib, sorafenib, sunitinib, vandetanib, and 33C3.

3. The compound mixture according to claim 1 wherein the inhibitor is cabozantinib or axitinib.

4. The compound mixture according to claim 1 wherein the inhibitor is cabozantinib.

5. The compound mixture according to claim 1, wherein compound b) is cabozantinib (S)-malate.

6. The compound mixture according to claim 1 wherein the inhibitor is axitinib.

7. A pharmaceutical composition, comprising;
a compound mixture according to claim 1 and, optionally, further comprising one or more excipient(s) and/or adjuvant(s).

8. A kit, comprising:
separate packs of compounds a) and b):
a) (S)-3-Hydroxy-1-(1H-indol-5-y1)-2-oxo-pyrrolidine-3-carboxylic acid 3,5-difluoro-benzylamide or a physiologically acceptable salt thereof; and
b) an inhibitor of one or more of VEGFR1, VEGFR2, VEGFR3_{.} and VEGF, or a physiologically acceptable salt thereof.

9. The kit according to claim 8, wherein the inhibitor is selected from the group consisting of aflibercept, apatinib, axitinib, bevacizumab, brivanib alaninate, cabozantinib, cediranib, lenvatinib, linifanib, pazopanib, ponatinib, ramucirumab, regorafenib, sorafenib, sunitinib, vandetanib, and 33C3.

10. The kit according to claim 8, wherein the inhibitor is cabozantinib or axitinib.

11. The kit according to claim 8 wherein the inhibitor is cabozantinib.

12. The kit according to claim 8 wherein compound b) is cabozantinib (S)-malate.

13. The kit according to claim 8 wherein the inhibitor is axitinib.

14. A method for prophylaxis or treatment of cancer, the method comprising;
administering to a subject compounds a) and b):
a) (S)-3-Hydroxy-1-(1H-indol-5-y1)-2-oxo-pyrrolidine-3-carboxylic acid 3,5-difluoro-benzylamide or a physiologically acceptable salt thereof; and
b) an inhibitor of one or more of VEGFR1, VEGFR2, VEGFR3, and VEGF, or a physiologically acceptable salt thereof.

15. The method according to claim 14, wherein the inhibitor is selected from the group consisting of aflibercept, apatinib, axitinib, bevacizumab, brivanib alaninate, cabozantinib, cediranib, lenvatinib, linifanib, pazopanib, ponatinib, ramucirumab, regorafenib, sorafenib, sunitinib, vandetanib, and 33C3.

16. The method according to claim 14, wherein the inhibitor is cabozantinib or axitinib.

17. The method according to claim 14, wherein the inhibitor is cabozantinib.

18. The method according to claim 14, wherein compound b) is cabozantinib (S)-malate.

19. The method according to claim 14, wherein the inhibitor is axitinib.

20. The method according to claim 14, wherein the cancer is a cancer for which the inhibitor is a standard-of-care treatment option and/or a cancer susceptible to anti-angiogenic treatments.

21. The method according to claim 14, wherein the cancer is selected from a group consisting of renal-cell carcinoma, colorectal cancer, lung cancer, head and neck cancer, gastric cancer, gastro-esophageal junction adenocarcinoma, gastrointestinal stromal tumors, glioblastoma, hepatocellular carcinoma, breast cancer, thyroid cancer, soft tissue sarcoma, chronic myeloid leukemia, and Philadelphia chromosome-positive acute lymphoblastic leukemia.

22. The method according to claim 14, wherein the cancer is renal cell carcinoma.

23. The method according to claim 22, wherein the renal cell carcinoma is clear cell renal cell carcinoma.

24. The method according to claim 14, wherein compounds a) and b) are administered simultaneously.

25. The method according to claim 14, wherein compounds a) and b) are administered sequentially.

26-40. (canceled)
